(19) United States
(12) Patent Application Publication
(10) Pub. No.: US 2012/0321688 A1
(43) Pub. Date: Dec. 20, 2012

(54) USE OF GLEDITSIA SAPONINS FOR CONTROLLING MOLLUSC PESTS

(75) Inventors: Xavier Crebassa, Bon Encontre (FR); Herve Caruel, Moncaut (FR); Frederic Mercier, Montagnac Sur Auvignon (FR); Ricardo Manuel Guillermo San Martin Gamboa, Providencia Santiago (CL)

(73) Assignee: DE SANGOSSE, Pont Du Casse (FR)

(21) Appl. No.: 13/576,200

(22) PCT Filed: Feb. 9, 2011
(86) PCT No.: PCT/FR2011/050270

§ 371 Date: Jul. 31, 2012

(30) Foreign Application Priority Data
Feb. 10, 2010 (FR) 1050921

(51) Int. Cl. A01N 45/00; A01N 63/00; A01N 25/08; A01P 9/00; C07H 15/256

(52) U.S. Cl. 424/409; 514/26; 536/5; 424/520

(57) ABSTRACT

Method of using saponins from at least one plant of the Gleditsia genus, or an extract of at least one plant of the Gleditsia genus including saponins, for controlling, by ingestion, land or aquatic mollusc pests. The molluscicidal active principle, compositions containing the same, and methods for controlling mollusc pests are also described.

This invention relates to the use of saponins obtained from plants of the genus Gleditsia as ingested poison for combating land or aquatic mollusk pests.

The invention also relates to a molluscicidal active agent that comprises Gleditsia saponins, as well as molluscicidal compositions that include such an active ingredient and their use as an ingested poison for combating land or aquatic mollusk pests.

Certain land or aquatic mollusks are known for being significant plant pests, able to damage agricultural, aquacultural, and horticultural products or amateur garden plots.

Certain land or aquatic mollusks are also known for having the capacity to transmit pathogens to humans, such as, for example, the aquatic snail Biomphalaria, and the slug of the genus Sarasinula.

So as to protect plants and crops from these pests and to prevent them from transmitting pathogens to humans, control measures must be put in place.

Different methods can be used to try to combat mollusk pests. The most common and the most effective method consists in resorting to molluscicidal products, i.e., active substances that have the property of killing mollusks.

Molluscicidal products are classified in two categories based on their mode of action: molluscicides that act by contact and those that act by ingestion.

The molluscicides that act by contact are those that, to be effective, have to come into direct or indirect contact with the mollusk pest. Their application can be carried out in various ways, and in particular by spreading powder or liquid solution in water, by direct spraying of a liquid solution on the mollusk pest, or by placing on the ground powder or impregnated strips containing the active ingredient.

The molluscicides by contact are used in particular to combat the aquatic mollusks, in particular in rice paddies. They are sprayed directly or spread in the water, and they expand throughout the entire aquaculture via the water that circulates in the network of irrigation canals. Since they are often toxic, they can cause water pollution and constitute a danger both for untargeted wildlife and for domestic animals and humans.

The molluscicides by contact are little used in combating land mollusk pests. Their major drawback resides in the fact that they are effective only if there is adequate physical contact between the molluscicidal product and the mollusk pest. If the mollusk does not come into contact with the molluscicide that is placed on the ground or does not come into contact adequately so that the lethal dose is reached, or else if the liquid solution is not sprayed at the exact moment when the infestation occurs, the pest is not eliminated and continues its ravages.

For these reasons, the ingested molluscicides are very widely preferred to the contact molluscicides for combating the land mollusk pests.

The molluscicidal active ingredients currently used are for the most part obtained from chemical synthesis. They act by ingestion and/or by contact with the mollusk.

The best known is metaldehyde, which acts by causing the lysis of the mucosal cells of mollusks up to the point where the mollusks die. The methiocarb that acts as a neurotoxin is also known.

These products of chemical origin are generally satisfactory in terms of effectiveness. However, the users are waiting for alternate solutions of natural origin, meeting in particular criteria of biological agriculture. To respond to this, various molluscicidal products of natural origin have already been developed. It involves in particular products that are obtained from plants or else microorganisms.

Among the molluscicides of natural origin that act by contact, it is possible to cite in particular the tea saponins, which are used in the rice paddies. A major drawback of the tea saponins resides in their high toxicity that damages the aquatic environment and untargeted animals. Experimental proceedings have also been conducted on the use of quinoa saponins as a contact molluscicidal poison for combating the aquatic snail known under the name of Golden Apple Snail.

There are also several natural products that have a molluscicidal effect by ingestion, but they often necessitate being used at very large doses to obtain an advantageous molluscicidal effect by ingestion, although it is technically very difficult, and even impossible, to formulate on an industrial scale molluscicidal bait based on these active ingredients of natural origin.

Also, the objective of this invention is to remedy the drawbacks of the prior art by proposing a product of natural origin that makes it possible to control in an effective manner by ingestion the land or aquatic mollusk pests, readily formulated in the form of bait on an industrial scale.

For this purpose, the invention has as its object the use of saponins that are obtained from at least one plant of the genus Gleditsia or an extract that is obtained from at least one plant of the genus Gleditsia that comprises saponins, for the control by ingestion of the land or aquatic mollusk pests.

Advantageously, the Gleditsia saponins are natural products, with a more favorable environmental profile than the molluscicides that are obtained from the chemical synthesis.

In a surprising way, they have a significant molluscicidal effect by ingestion both relative to the land mollusks and to aquatic mollusks, even at low concentration.

The invention also has as its object a molluscicidal active ingredient that comprises Gleditsia saponins, as well as a molluscicidal composition that integrates such an active ingredient.

Finally, the purpose of this invention is also a process for combating land mollusk pests by ingestion and a process for combating aquatic mollusk pests by ingestion comprising the application of a molluscicidal composition that integrates Gleditsia saponins.

Other characteristics and advantages will emerge from the following description in detail of the invention.

According to a first aspect, the invention therefore relates to the use of saponins that are obtained from at least one plant of the genus Gleditsia or an extract that is obtained from at least one plant of the genus Gleditsia that comprises saponins, for monitoring by ingestion mollusk pests, i.e., for combating land or aquatic mollusk pests.

In particular, the purpose of the invention is the use of saponins that are obtained from at least one plant of the genus Gleditsia or an extract that is obtained from at least one plant of the genus Gleditsia as a molluscicidal active agent in a composition that is designed to be ingested by land or aquatic mollusk pests.

If an extract of Gleditsia is involved, the latter is preferably a purified extract from at least one plant of the genus Gleditsia that comprises at least 20% of saponins by weight.

In a preferred way, the saponins are at least in part triterpenic saponins. They have an even better molluscicidal effectiveness.

The saponins or the extracts that comprise saponins are obtained from one or more plants that belong to the genus Gleditsia, such as, in particular, Gleditsia amorphoïdes, Gleditsia aquatica, Gleditsia australis, Gleditsia delavayi, Gleditsia fera, Gleditsia japonica, and Gleditsia rolfei.

According to a particularly suitable embodiment, the saponins or the extract that contains the saponins are obtained from at least one plant of the family of Gleditsia of the species amorphoïdes.

According to a variant, the Gleditsia saponins can be at least partially hydrolyzed, according to any suitable process. A suitable process can consist of, for example, the implementation of the following stages:
Immersing a plant fraction that contains Gleditsia saponins in water at a high temperature until saponins are extracted,
Optionally renewing the first stage multiple times so as to maximize the amount of Gleditsia saponins that are recovered,
Separating the fraction of the plant from the liquid solution that contains the saponins by decanting or filtering, for example,
Adding a basic agent (pH>7) into the liquid solution and heating the batch to carry out a hydrolyzation of the saponins,
Adding an acid agent (pH<7) into the liquid solution to initiate its neutralization, and
Optionally dehydrating the thus obtained solution so as to obtain a powder, by any suitable process, such as, for example, atomization.

The invention is effective against any land or aquatic mollusk pests. It is very particularly effective against:
Slugs of the families Limacidae, Arionidae, Milacidae, more particularly the species Deroceras Reticulatum and Anion Hortensis,
Land snails of the genera Helix, Discus, Euomphalia, and more particularly the species Helix Aspersa, and Theba pisana,
Aquatic snails of the genera Biomphalaria, Lymnea, Pomacea, and more particularly Pomacea canaliculata.

According to the invention, the Gleditsia saponins or the extracts that comprise Gleditsia saponins act by ingestion. They can be used integrated in different substrates that are suitable for this mode of action.

The Gleditsia saponins or the extracts that comprise the Gleditsia saponins can be used in particular in bait in the form of pellets, powder, or a powdery, block or gel mixture, or a coated neutral substrate mixture.

Advantageously, the Gleditsia saponins or the Gleditsia extracts that comprise saponins, in particular Gleditsia triterpenic saponins, have a molluscicidal effect even at very low concentrations. These saponins or extracts are therefore readily formulated on an industrial scale. In addition, the use of Gleditsia saponins at low concentration has another advantage. Actually, because of their physico-chemical and emulsifying properties, saponins, if present in too large a quantity in the bait, can ultimately have a repellant effect on mollusk pests, which thereby do not consume them and are therefore not eliminated. Since they are effective at low concentration, the Gleditsia saponins make it possible to avoid these problems of partiality.

The Gleditsia saponins or the extract comprising such saponins can be used alone or in combination with other molluscicidal components.

According to a second aspect, the invention relates to a molluscicidal active ingredient that comprises saponins obtained from at least one plant of the genus Gleditsia.

A preferred species of Gleditsia is the amorphoïdes species.

Preferably, the active ingredient contains at least 20% Gleditsia saponins, expressed in terms of weight of the dry extract.

According to a particularly suitable embodiment, the saponins are at least in part triterpenic saponins.

The Gleditsia saponins or plant extracts that contain the Gleditsia saponins can be obtained by solid-liquid extraction, preferably by aqueous extraction, followed by a purification and concentration stage, preferably carried out by membrane ultrafiltration. Any part of the plant that contains saponins can be used. Nevertheless, the shells of fruits that are particularly rich in saponins will be preferred.

The active ingredient according to the invention can be integrated in a composition that is designed for combating land mollusk pests by ingestion or a composition that is designed to combat aquatic mollusk pests by ingestion.

This composition comprises between 2 and 20% of the active ingredient by weight of the composition, preferably between 5 and 10%.

The purpose of the invention is also a composition that comprises between 0.1 and 10% of Gleditsia saponins by weight of the composition, preferably between 1 and 5%.

The composition can also comprise additional ingredients that are commonly used, such as, for example, meal, grain co-products, preservatives or attractive agents.

It can also comprise one or more other molluscicidal active ingredients such as, by way of nonlimiting example, metaldehyde, methiocarb, carbaryl, a ferric compound or complex, nematodes, biological toxins, or saponins that are obtained from plants other than Gleditsia, having a molluscicidal effect by ingestion.

The composition according to the invention is designed to act by ingestion. It can come in the form of pellets, powder, or a powdery, block or gel mixture, or a coated neutral-substrate mixture or any other form that is suitable for combating land or aquatic mollusk pests by ingestion. It generally consists of or is combined with an edible, appetizing substrate for the mollusks.

A coating of the active ingredient optionally can be made using suitable complementary techniques and ingredients. This coating makes it possible in particular to mask the presence of saponins. By way of nonlimiting example, these ingredients can be selected in the family of acrylic derivatives, vinyl derivatives, cellulose derivatives or gums. However, this coating, if it is preferred, is not necessary because the saponins can be used at a low enough concentration to prevent an impact on the palatability of the bait.

The composition can be obtained by simple mixing of the components or by processes that are known to one skilled in the art.

One example of a composition for a land or aquatic mollusk that is designed to be ingested can consist of the following elements:
70 to 85% of an appetizing substrate that comprises grain meal and other grain co-products,
2 to 20% of an active ingredient according to the invention that comprises at least 20% of Gleditsia saponins,
0.01 to 0.1% of a dye,
0.2 to 2% of a preservative of the organic acid type and salts thereof such as sorbic acid, citric acid, or sodium benzoate,
0.5 to 5% of appetizing additives such as sugar, sweeteners, surfactant phagostimulants, products obtained from yeast, and milk proteins,
Additives that make it possible to limit the crumbling of the bait such as hydrocolloids, paraffin or egg proteins, and
0.005 to 0.05% of an agent that makes it possible to reduce the risk of accidental consumption by children or domestic animals such as denatonium benzoate.

Such a composition corresponds to a molluscicidal bait in pellet or block form.

This product can be manufactured in particular by the implementation of the following stages:
Weighing the different ingredients,
Mixing the ingredients using a ribbon mixer or a high-power mixer,
Incorporating an amount of water in vapor form or in liquid form and/or melted paraffin so as to form a continuous homogeneous mixture,
Extrusion or compression of this mixture through a die using an extrusion cooker or else a high-pressure press,
Cutting the pieces of bait,
Stabilization of the bait by cooling/drying so as to allow an at least two-year storage, and
Packaging of the bait in packages that are suitable for use.

According to a last aspect, the objects of the invention are processes for combating mollusk pests.

A first process that is designed to combat land mollusks consists in spreading molluscicidal compositions according to the invention (also called bait) manually or mechanically preferably using a centrifugal spreader on the ground when the infestation is underway or else preventively when the climatic conditions make it possible to reasonably predict that this infestation will occur. The bait preferably has the shape of cylindrical granules. Several passages may be necessary based on climatic conditions and infestation level. In any case, the program of attack is to be conducted in a responsible manner, and, if it is designed to protect large crops, with respect to good agricultural practices.

The bait can also be mixed with seed. It is then spread at the same time as the latter, which can make it possible to make only a single application and therefore to streamline the costs.

The bait can also be placed on or in the furrow with a micro-granulator-type device, for example.

A second process that is designed for combating aquatic mollusks consists in spreading, manually or mechanically, the molluscicidal compositions according to the invention (or bait), preferably having the shape of cylindrical granules, in the region of water to be treated, or else, if it is bulkier, to place this bait on substrates that are embedded in the ground of the aquaculture, making it possible to keep the bait above the silt and below the surface of the water, with this system making it possible to keep the bait from being embedded in the silt and therefore promoting its consumption by aquatic mollusk pests.

The invention is now illustrated by test results showing the claimed effects.

### I. EVALUATION OF THE MOLLUSCICIDAL EFFECT OF GLEDITSIA SAPONINS

The objective of this study is to show the molluscicidal effect of Gleditsia saponins.

The operating procedure is described below.

Gray slugs (Deroceras reticulatum) of homogeneous adult size (slugs collected on the same day on the same sampling site) are collected in an open field and kept under breeding conditions: temperature between 10 and 20° C., relative humidity of between 60 and 90% with a photoperiod in a plastic box.

The diet of the slugs consists exclusively of plants.

A water-soaked filter paper is placed at the bottom of the plastic box. A plastic substrate that they use as a refuge zone is placed at the center of the box.

For carrying out tests, a box that contains slugs is connected to a CO₂ bottle. A controlled stream of CO₂ is administered to the slugs for several minutes before anesthetizing them.

The slugs are next handled in such a way that they ingest a hydrocolloid-based aqueous gel, containing Gleditsia amorphoïdes saponins. The percentages that are mentioned in the table below express the pure saponin content.

A placebo control gel is also tested.

For each test, 10 slugs are used.

The slugs are next put into a breeding box, and the number of dead slugs is noted at the end of 4 days.

The results that are obtained are presented in Table 1 below:

| | Percentage of Dead |
|---|---|
| | Slugs on D + 4 (in %) |
| Placebo Control | 0 |
| Placebo Control | 0 |
| 0.5% Gleditsia [symbol] Saponins | 10 |
| 0.5% Gleditsia [symbol] Saponins | 10 |
| 1% Gleditsia [symbol] Saponins | 40 |
| 1% Gleditsia [symbol] Saponins | 50 |
| 2% Gleditsia [symbol] Saponins | 100 |
| 2% Gleditsia [symbol] Saponins | 80 |
| 2% Gleditsia [symbol] Saponins | 90 |
| 2% Gleditsia [symbol] Saponins | 100 |
| 2% Gleditsia [symbol] Saponins | 70 |
| 2% Gleditsia [symbol] Saponins | 70 |

These results clearly show that the Gleditsia saponins have a molluscicidal effect by ingestion. The effects are significant starting from 1% of pure gel-formulated saponins.

### II. EVALUATION OF THE MOLLUSCICIDAL EFFECT OF GLEDITSIA SAPONINS IN COMPARISON WITH OTHER MOLLUSCICIDAL PRODUCTS

The objective of this study is to evaluate the molluscicidal effect by ingestion of Gleditsia saponins in comparison to that of other saponins that have a molluscicidal effect.

The operating procedure is identical to that of the preceding point I.

The hydrocolloid-based aqueous gel contains the following active ingredients that are to be tested:
A: Gleditsia amorphoïdes saponins
B: Saponins that are obtained from quinoa powder
C: Saponins that are obtained from quinoa, having undergone an alkaline treatment
D: Saponins that are obtained from tea powder
E: quillaja saponins, enough to produce 100
F: quillaja saponins, enough to produce 1,000
G: Yucca saponins
H: Chenopodium album saponins.

For each product, 10 slugs are used.

After this stage, the slugs are put in a breeding box.

The number of dead slugs is next noted each day for a week so as to determine the lethal dose 50 (LD₅₀) at 3 days of each of the products tested.

The results that are obtained are presented in Table 2 below:

| Tested Extracts | LD₅₀ 3 Days in μg/Slug |
|---|---|
| A (Invention) | 90 |
| B | >400 |
| C | 230 |
| D | 180 |
| E | 250 |
| F | 190 |
| G | 230 |
| H | >270 |

These results confirm that the Gleditsia (A) saponins clearly exhibit a molluscicidal effect. In addition, it is noted that this effect is better than that which is obtained with other saponins that have a molluscicidal effect by ingestion, and can be used at much lower concentrations.

### III. EVALUATION OF THE MOLLUSCICIDAL EFFECT OF A COMPOSITION ACCORDING TO THE INVENTION

The objective of this study is to show the molluscicidal effect by ingestion of a composition that contains Gleditsia saponins.

The tests are carried out under controlled conditions on the aquatic snail Golden Apple Snail (Pomacea Canaliculata). Wild aquatic snails are collected in the natural medium (irrigation channels) and stored in drums. A period of acclimation of one week in duration is then complied with so as to validate the good state of health of the collected snails.

The snails are then introduced into suitable water-filled beakers at a rate of one snail per beaker before the tests are carried out.

Several pieces of bait, in the form of granules and containing different molluscicidal active ingredients by ingestion, are tested.

The pieces of bait that are used for this test in pellet or block form have the following composition:
Sufficient quantity of an appetizing substrate that comprises grain meal and other grain co-products,
A variable percentage of active ingredients A to K,
0.05% of blue dye PB15,
0.005% of denatonium benzoate,
3% sugar,
0.5% of carrageenans.

This composition is obtained by implementing the following stages:
Weighing the different ingredients,
Mixing the ingredients using a ribbon mixer,
Incorporating water in the form of vapor so as to form a continuous homogeneous mixture,
Extrusion of this mixture through a die using a high-pressure press,
Cutting the pieces of bait, and
Stabilization of the bait by cooling/drying.

The tested active ingredients are as follows:
A: 5% metaldehyde
B: 1% quinoa saponins
C: 5% quillaja saponins
D: 1% quillaja saponins
E: 1% quinoa saponins that have undergone an alkaline treatment
F: 0.1% tea saponins
G: 1% tea saponins
H: 0.5% yucca saponins
I: 0.1% Gleditsia amorphoïdes saponins
J: 0.5% Gleditsia amorphoïdes saponins
K: 1% Gleditsia amorphoïdes saponins.

The percentages that are expressed above are percentages of active ingredient. For each product, 10 snails are used.

The number of dead snails is recorded each day for a period of 5 days.

The results that are obtained are presented in Table 3 below.

| Tested Active | % Cumulative Mortality |
|---|---|
| Ingredients | Golden Apple Snail D + 5 |
| A | 80 |
| B | 10 |
| C | 40 |
| D | 0 |
| E | 30 |
| F | 0 |
| G | 40 |
| H | 20 |
| I (Invention) | 60 |
| J (Invention) | 60 |
| K (Invention) | 30 |

These results confirm that a composition that contains Gleditsia saponins (I, J and K) is consumed by a pest and has a molluscicidal effect by ingestion. In addition, it is noted that this effect is significant, even at a low concentration.

1. A process for combating land or aquatic mollusk pests comprising administering to said mollusk pests by ingestion saponins that are obtained from at least one plant of the genus Gleditsia, or an extract that is obtained from at least one plant of the genus Gleditsia that comprises saponins.

2. The process according to claim 1, wherein the administration of Gleditsia saponins or the extract that is obtained from Gleditsia that comprises saponins is as an ingested molluscicidal active agent in a composition that is designed to be ingested by land or aquatic mollusk pests.

3. The process according to claim 1, wherein the saponins are at least in part triterpenic saponins.

4. The process according to claim 1, wherein the saponins or the extract that comprises saponins are obtained from at least one plant of the genus Gleditsia of the species amorphoïdes.

5. Molluscicidal active ingredient, comprising saponins obtained from at least one plant of the genus Gleditsia.

6. Molluscicidal active ingredient according to claim 5, wherein it is an extract from at least one plant of the genus Gleditsia that comprises at least 20% of saponins by weight.

7. Molluscicidal active ingredient according to claim 5, wherein the saponins are at least in part triterpenic saponins.

8. Molluscicidal active ingredient according to claim 5, wherein the saponins are obtained from at least one plant of the genus Gleditsia of the species amorphoïdes.

9. Composition that is designed for combating land or aquatic mollusk pests, wherein it comprises between 0.1 and 10% of Gleditsia saponins or between 2 and 20% of an active ingredient according to claim 5 and an edible appetizing substrate for the mollusks.

10. Composition that is designed for combating land or aquatic mollusk pests according to claim 9, wherein the active ingredient is coated with an ingredient that is obtained from the family of acrylic derivatives, vinyl derivatives, cellulose derivatives or gums.

11. Molluscicidal composition that is designed for combating land or aquatic mollusk pests according to claim 8, wherein it comes in the form of pellets, powder, or a powdery, block or gel mixture, or a coated neutral substrate mixture or any other form that is suitable for combating land or aquatic mollusk pests by ingestion.

12. Molluscicidal composition that is designed for combating land or aquatic mollusk pests according to claim 8, wherein it also comprises at least one other active ingredient that has a molluscicidal effect.

13. Molluscicidal composition that is designed for combating land or aquatic mollusk pests according to claim 12, wherein it also comprises at least one other active ingredient that has a molluscicidal effect that is selected from among metaldehyde, methiocarb, carbaryl, a ferric compound or complex, nematodes, biological toxins, or saponins that are obtained from plants other than Gleditsia, having a molluscicidal effect.

14. Process for combating land mollusk pests, wherein it consists in manually or mechanically spreading a composition on the ground according to claim 8.

15. Process for combating aquatic mollusk pests, wherein it consists in manually or mechanically spreading a composition in a region of water to be treated according to claim 8.

16. Process for combating aquatic mollusk pests, wherein it consists in placing a composition according to claim 8 on substrates that are embedded in the ground, making it possible to keep the bait above ground level and below the surface of the water.
